(19) United States
(12) Patent Application Publication
(10) Pub. No.: US 2021/0012878 A1
(43) Pub. Date: Jan. 14, 2021

(54) SYSTEMS AND METHODS FOR RADIATION TREATMENT PLANNER TRAINING BASED ON KNOWLEDGE DATA AND TRAINEE INPUT OF PLANNING ACTIONS

(71) Applicants: Duke University, Durham, NC (US); The University of North Carolina at Charlotte, Charlotte, NC (US); The United States Government as represented by the Department of Veterans Affairs, Washington, DC (US)

(72) Inventors: Qingrong Wu, Matthews, NC (US); Yaorong Ge, Matthews, NC (US); Fang Fang Yin, Durham, NC (US); Matthew Mistro, Durham, NC (US); Yang Sheng, Durham, NC (US); Christopher Kelsey, Durham, NC (US); Joseph Salama, Durham, NC (US)

(21) Appl. No.: 16/926,611

(22) Filed: Jul. 10, 2020

Related U.S. Application Data
(60) Provisional application No. 62/872,294, filed on Jul. 10, 2019.

(51) Int. Cl. G16H 20/40; G16H 70/20; G16H 40/20; G16H 30/20

(52) U.S. Cl. CPC G16H 20/40; G16H 30/20; G16H 40/20; G16H 70/20

(57) ABSTRACT

Systems and methods for radiation treatment planner training based on knowledge data and trainee input of planning actions are disclosed. According to an aspect, a method includes receiving case data including anatomical and/or geometric characterization data of a target volume and one or more organs at risk. Further, the method includes presenting, via a user interface, the case data to a trainee. The method also includes receiving input indicating an action to apply to the case. The input indicating parameters for generating a treatment plan that applies radiation dose to the target volume and one or more parameters for constraining radiation to the one or more organs at risk. The method includes applying training models to the action and the case data to generate analysis data of at least one parameter of the action. The method includes presenting the analysis data of the at least one parameter of the action.

### CROSS REFERENCE TO RELATED APPLICATION

This application claims priority to U.S. Patent Application No. 62/872,294, filed Jul. 10, 2019, and titled SYSTEMS AND METHODS FOR IMPROVING THE PLANNING KNOWLEDGE OF RADIATION TREATMENT PLANNERS, the content of which is incorporated herein by reference in its entirety.

### FEDERALLY SPONSORED RESEARCH OR DEVELOPMENT

This invention was made with government support under Federal Grant No. R01CA2020, awarded by the National Institutes of Health (NIH). The government has certain rights to this invention.

### TECHNICAL FIELD

The presently disclosed subject matter relates generally to radiation therapy planning training and medical education. Particularly, the presently disclosed subject matter relates to training of clinical staff for radiation treatment planning based on knowledge data and trainee input of planning actions.

### BACKGROUND

Radiation therapy, or radiotherapy, is the medical use of ionizing radiation to control malignant cells. Radiation treatment planning is the process in which a team of clinical staff consisting of radiation oncologists, medical physicists and medical dosimetrists (also referred to as “treatment planners”) plan the appropriate external beam radiotherapy or internal brachytherapy treatment for a patient with cancer. The design of a treatment plan, including a set of treatment parameters, aims to reach an optimal balance of maximizing the therapeutic dose to the tumor target (i.e., plan target volume or PTV) while minimizing the dose spills (i.e., radiation induced toxicity) to the surrounding Organs-At-Risk (OARs). Radiation treatment planning involves complex decision making in specifying optimal treatment criteria and treatment parameters that take into account all aspects of patient geometry and conditions and treatment constraints and also in utilizing the most appropriate optimization algorithms and parameters to reach an optimal treatment plan.

Multiple studies have shown planner experience playing an important role in affecting plan quality, especially in difficult cancer sites and scenarios. One approach to improving the quality and efficiency of radiation treatment planning is to develop artificial intelligence (AI) techniques such as knowledge models and even automated planning algorithms so that planners with less experience or at smaller treatment centers can leverage the knowledge of more experienced planners. Various technologies have been developed in this area. However, artificial intelligence-based techniques are unlikely to replace human decisions in radiation therapy due to the complexity of patient conditions and the inherent conflicting nature of cancer control and normal tissue sparing. As long as human decisions are required, a solid understanding of principles behind treatment planning as well as the knowledge models and artificial intelligence techniques that are developed to support treatment planning are important to improve decision making.

In addition to traditional techniques of medical education and training such as classes and workshops, numerous learning and tutoring technologies have been developed to enhance the process and experience of learning medical diagnosis and various complex procedures. These existing systems typically involve multiple iterations of three components: present a scenario (e.g., a case description, a video segment, or a physical simulator) to the trainee; request an answer (e.g., a diagnosis, a next step, or an action); and provide an assessment (e.g., a correct/incorrect response or a simulator reaction). Two common themes that underlie all the existing training systems are that: the knowledge to be transferred is static, deterministic, and binary (i.e., either correct or wrong); the goal is to teach how and what to do something rather than why something is correct or preferable.

With regard to radiation therapy planning, the decision process is complex and involves difficult trade-offs at every step. Possible actions at each step are rarely either correct or incorrect. In fact, what is important for trainees to learn are often good strategies rather than clear cut instructions and concrete understandings rather simple next steps. Existing training techniques do not address these challenges. Therefore, there is a continuing need for improved systems and techniques for training in the field of radiation therapy planning.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a block diagram of a system for radiation treatment planning based on knowledge data and trainee input of planning actions in accordance with embodiments of the present disclosure;

FIGS. 2A and 2B are graphs showing trainee scores for a benchmark plan and five plans according to the present disclosure, respectively;

FIG. 3 is a block diagram of an example training system in accordance with embodiments of the present disclosure;

FIG. 4 is a flow diagram showing an example training workflow for learning to plan a training case in accordance with embodiments of the present disclosure;

FIG. 5 is a flow diagram of an intelligent learning method;

FIG. 6 is a block diagram of an intelligent learning system in accordance with embodiments of the present disclosure;

FIG. 7 is a graph depicting knowledge improvements after use of the intelligent learning program;

FIG. 8 are images that illustrate gDTH, which is a 2D feature map when there is only 1 primary and 1 boost PTV volume, and becomes high dimensional with additional volumes;

FIG. 9 illustrates a graph and images showing interpretation and visualization of gDTH for left parotid;

FIG. 10 illustrates different images showing beam configuration and a dose-volume histogram; and

FIG. 11 is a block diagram of an example system for radiation treatment planner training based on knowledge data and trainee input of planning actions in accordance with embodiments of the present disclosure.

### SUMMARY

The presently disclosed subject matter relates to systems and methods for radiation treatment planner training based on knowledge data and trainee input of planning actions. According to an aspect, a method includes receiving case data including anatomical and/or geometric characterization data of a target volume and one or more organs at risk. Further, the method includes presenting, via a user interface, the case data to a trainee. The method also includes receiving, via the user interface from the trainee, input indicating an action to apply to the case, the input indicating one or more parameters for generating a treatment plan that applies radiation dose to the target volume and one or more parameters for constraining radiation to the one or more organs at risk. Further, the method includes applying training models to the action and the case data to generate analysis data of at least one parameter of the action. The method also includes presenting, via the user interface, the analysis data of the at least one parameter of the action.

According to another aspect, a method includes receiving case data comprising subject disease information, other medical condition information, and/or anatomical and/or geometric characterization data of a target volume and one or more organs at risk of a subject. The method also includes providing identification of one or more treatment plan analysis points for the subject. Further, the method includes providing knowledge data about treatment of the subject at the one or more treatment plan analysis points. The method also includes presenting, via a user interface, the case data to a trainee and the identified one or more treatment plan analysis units, and questions regarding the units. Further, the method includes receiving, via the user interface from the trainee, response input to presentation of the one or more treatment plan analysis points. The method also includes generating analysis data of trainee feedback based on knowledge data and the response input in response to receipt of the response input. Further, the method includes presenting the analysis data via the user interface to the trainee in response to receipt of the response input.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or times program instructions thereon for causing a processor to carry out aspects of the present disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

In accordance with embodiments, systems and methods are provided for training radiation treatment planners or trainees. These systems and methods can be used to guide the trainees toward good planning strategies and concrete understandings. An important example aspect to this technique is the provision of a set of knowledge models and other artificial intelligence techniques that predict the desired result in various scenarios and a set of evaluation metrics that can elicit good understanding of reasons behind good versus bad plans. As an example, as the trainee is guided by the system through multiple steps of radiation treatment planning, the trainee can iteratively acquire improved strategies and understanding of the physics and optimization nature of radiation treatment by comparing his or her own results with the model predicted results as well as examining the difference in various evaluation metrics.

In accordance with embodiments, training systems and methods disclosed herein can enable planners with various levels of experience to improve their knowledge in intensity modulated radiation therapy (IMRT)/volumetric modulated arc therapy (VMAT) planning with different levels of complexity. For example, the systems and methods involve methods to systematically address the needs for selecting optimized beam configurations and models, and for performing guided tradeoffs all with the help of various knowledge models including, but not limited to, a beam angle selection model and a dose-volume histogram (DVH) prediction model.

In accordance with embodiments, a beam model can operate based on an efficiency index that functions to maximize dose delivered to a PTV and to minimize the dose delivered to OARs based on a number of learned weighting factors. Further, the beam model can induce a forced separation between good quality beams in the efficiency index to not lose out on conformality. For the purpose of simplicity of introduction to new planners, all beams can be restricted to co-planar. Users can select the number of beams needed.

In accordance with embodiments, a DVH prediction model can be provided. The DVH prediction model can utilize principal component analysis of a number of anatomical features: distance-to-target histogram (DTH) principal components, OAR volume, PTV volume, OAR-PTV overlapping volume, out-of-field OAR volume, and the like. In experiments, a DVH prediction model was trained with 74 lung cases with a variety of tumor sizes and locations, and it can be used to predict DVHs for: cord, cord+3 mm, lungs, heart, and esophagus. As the model is dependent on the out-of-field OAR volume, it may be dependent on the beam selection. The predictions were made based on the clinically delivered plan's beam selection, and an assumption was made that the trainee's beams were close enough to the beams that the clinically delivered plan was built upon. While that assumption may not always hold true, it is typically clear that when the trainee's beams differ by a large amount it results in an inferior plan.

In an example use of a system as disclosed herein, each trainee can be provided with the scoring metrics and can be asked to plan an intermediate-to-hard difficulty plan without any intervention from the trainer, knowledge-based models, or user interface. The difficulty can be determined by looking at the prescription, tumor size, complexity of shape, and proximity to OARs. The trainee can be instructed that he or she has a choice between a 6 and 10 MV beam and can have no more than 11 beams to align with current clinical practice.

After the benchmark of intermediate-difficult rating with a target volume of 762.8 cm³ and a prescription of 62 Gy, the system involved 5 additional patients: two easier cases with an average target volume of 113.8 cm³ and prescriptions of 40 Gy, one intermediate case with a target volume of 453.0 cm³ and a prescription of 60 Gy, and two difficult cases with an average target volume of 845.7 cm³ and prescriptions of 60 Gy. A user interface may be presented for a comparison of two plans with scores, DVHs, predicted DVHs, and objective constraints is supplied for use for the training directly the system. Interaction can be on the basis of the trainee attempting to explain differences between their plan and a comparison plan and the resulting dosimetric implications of those differences. The comparison plan can change based on where he or she is within the system. This comparison plan can start as a plan with varied inputted beams from the beam model. This can start as seven beams for an intermediate comparison and can end with 11 beams for the final. The trainee can evaluate whether she or he wants to move in the direction of the model or continue with her or his own direction, and the final geometric comparison she or he makes is an assessment on what the dosimetric differences in their plan versus the comparison can be based on geometry. After the beam setting is determined, the trainee can create initial optimization constraints and the constraints can be copied into the knowledge-based beam plan, and a dosimetric comparison is implemented to check whether the results aligned with the trainee's expectation which builds a forward intuition on beam choice implications. Following this, the DVH model can be imported and the trainee is able to compare end DVHs and dose objectives to where the DVH model predicts the trainee is capable of achieving and they make changes based on what they see is obtainable. After the changes are made, the plan can be scored, and the final comparison plan is the clinically delivered plan. The trainee can work backward by looking at the scoring and DVHs of the clinical plan and can make a judgement on how the trainee thinks the clinically delivered plan was set up. This is to further ingrain a backwards intuition for the metrics related to certain collective beam arrangements.

In embodiments, there can be two different points in training where no more changes may be made to that portion of the plan, right before making the final comparison the beam model and the iteration after making changes from what they see is what may be expected. This can serve to cut back on constant iteration for small improvements and force more careful thought in the trainee's initial attempts. Although, it can be realized from a trial trainee that the more difficult plans may need more iteration, so the pipeline for the last three plans gives opportunity to change the beam selection after the dosimetric comparison between the trainee's plan and the beam model plan.

For people with little to no knowledge of treatment planning, the first beam assessment can be a review session with the trainer module of the system to understand if he or she was to make a plan with only a single beam, which direction would be best, and this tries to get them to think of how each individual beam may contribute. The second beam assessment session will review how to make an optimal plan if only two beams are used, and this starts to instill in the trainee how the beams may be interacting with one another (i.e., the second absolute best beam may not necessarily be the best beam to work with the first). An additional basic constraint assessment step checks to ensure that the trainee has at least one objective for all the relevant structures and two for the target.

Following at least five training case plans, the trainee can return to the same patient case that he or she did the baseline on and plan it again to see if there was any significant improvement in their planning ability. This can be done without any intervention from the trainer module, knowledge-based models, or user interface.

In accordance with embodiments, a scoring system is provided to facilitate communication of comparison between different plans and the metrics with their respective maximum point value. These systems can be constructed in various ways, but this example starts with the logic of rewarding dosimetric endpoints that are clinically relevant as starting with RTOG reports, other clinical considerations and planning competitions. A conformality index (CI) can function to limit the isodose volume and can be represented as:

$${CI}_{x \%} = \frac{{V}_{x \%}}{{V}_{PTV}} \qquad ( 1 )$$

The conformation number (CN) or Paddick conformity index can follow similar logic as applied to CI but focuses on the portion of that isodose volume within the target, as set forth in the following equation:

$${CN}_{x \%} = \frac{{( {V}_{x \%}^{PTV} )}^{2}}{{V}_{PTV} \times {V}_{x \%}} \qquad ( 2 )$$

Within a scoring system such as this, knowing the metrics and priorities can provide some information on how to pursue the problem at hand. Unfortunately, this may not always hold true, so the scoring may be adjusted to hold a second function of having “guiding metrics” to lead towards clinically feasible plans. An example of this is V_{5Gy} versus V_{20Gy} of the lung. Physicians may give more priority to V_{20Gy} as an indicator of pneumonitis, but the nature of V_{5Gy} being more sensitive to careful beam selection can mean a better guidance to how a person selects their beams. V_{20Gy} can be forced in an optimization without causing other metrics to be entirely infeasible, but the plan may not necessarily be as good as it would have with more meaningful beam selection. The guiding component is to weigh the priority of V_{5Gy} higher, so the trainee puts priority on careful beam selection without producing a degenerate plan.

It is noted that in the aforementioned some of the aforementioned examples and embodiments, the trainer module refers to an intelligent tutoring agent, but more generally it can also be a human trainer in some instances, for example, when the intelligent tutoring agent is insufficient. Also, the number and selection of patient cases, beam angles, and knowledge models can vary depending on training settings and trainee characteristics.

In summary, systems and methods disclosed herein can be used to ensure that the treatment planning knowledge extracted from prior plans and put into practice are not only contained in knowledge models and planning systems (like robots), but are also communicated to planners and physicians. Hence, by provision of systems and methods disclosed herein, planners and physicians, experienced or not experienced, can have readily available training systems to elevate them to the expert level of treatment planning with full confidence in leveraging knowledge models and other AI tools.

In an example use case, knowledge-based models can be used to train human planners or trainees with lung and media stinum IMR planning. Particularly, a purpose in this case can be to develop an intelligent learning system by incorporating knowledge-based treatment planning models to serve as informative, efficient bases to train a person to plan particular sites while creating confidence in utilizing these models in a clinical setting. In methods, a beam angle selection model and a DVH prediction model for lung/mediastinum IMRT planning can be used as the information centers within a directed training system guided by scoring criteria and communicated with the trainees via a GUI ran from the Eclipse TPS. The scoring can serve both to illustrate relative quality of plans and as a guide to facilitate directed changes within the plan. A mediastinum patient can serve as a benchmark to show skill change from the intelligent learning system and is completed without intervention. Five additional lung/mediastinum patients follow in the subsequent training pipeline where the models, GUI and trainer work with trainee's directives and guide meaningful beam selection and tradeoffs within IMRT optimization. Five trainees with minimal treatment planning background were evaluated by both the scoring criteria and a physician to look for improved planning quality and relative effectiveness against the clinically delivered plan. As a result, trainees scored an average of 22.7% of the total points within the scoring criteria for their benchmark and returned to improve to an average of 51.9% compared to the clinically delivered of 54.1%. Two of the five trainee final plans were rated as comparable to the clinically delivered by a physician and all five noticeably improved. For plans within the system, trainees performed on average 24.5% better than the clinically delivered plan with respect to the scoring criteria.

In another example use case, knowledge-based models can be used in an intelligent learning system for lung IMRT planning. In this case, the system can be constructed around two knowledge-based treatment planning models. This system can be used to quickly bring unexperienced planners to a clinically feasible level while building a foundation of trust in utilizing these knowledge-based models as supplementary tools within the clinic. Further, this system is moving into a regime of human focused AI in treatment planning where one does not preclude the other. It is noted that knowledge-based treatment planning models can serve as informative evidence to guide IMRT. However, this AI tool behaves as a “black box” to the majority of users and does not show the capability of improving the skill level of human planner. In this study, one goal is to use knowledge-based models to train individuals to develop IMRT plans for a particular site while building confidence in utilizing these models in a clinical setting and moving towards human-centered AI.

Continuing the aforementioned use case, a beam angle selection model and a DVH prediction model for lung/mediastinum IMRT planning are used as the information centers within a directed intelligent learning system guided by as structured scoring criteria. The intelligent learning system can communicate with trainees via a scripting user interface in the treatment planning system. A total of six lung/mediastinum IMRT patients' plans were built in the system. One patient can serve as a benchmark to show skill development from the intelligent learning system and is completed without intervention. Five additional lung/mediastinum patients follow in the subsequent training pipeline where the models, GUI and trainer work with trainee's directives and guide meaningful beam selection and tradeoffs within IMRT optimization. Five trainees with minimal treatment planning background were trained through the entire training pipelines and subsequently evaluated by both the scoring criteria and a radiation oncologist to look for longitudinal planning quality improvement and relative effectiveness against the clinically delivered plan. As a result, trainees scored an average of 22.7% of the total points within the scoring criteria for the lone benchmark plan yet improved to an average of 51.9% compared to the clinically delivered plan which achieved 54.1% of the total potential points. Two of the five trainee final plans were rated as comparable to the clinically delivered by a physician and all five were noticeably improved by the physician's standards. For the other five plans within the system, trainees performed on average 24.5% better than the clinically delivered plan with respect to the scoring criteria. Based on this use case and its result, it can be understood that the system can bring unexperienced planners to a level comparable of experienced dosimetrists for a specific treatment site and when used to inform decisions.

In accordance with embodiments, systems and methods disclosed herein can use knowledge-based models to collect and summarize information from previous plans and utilize them to build model capable of predicting clinically relevant solutions. For treatment planning, this can come in the form of generated beam angles, optimized collimator settings, predicted achievable dose-volume histogram (DVH) endpoints for inverse optimization, or a combination thereof for a fully automated treatment planning process. Knowledge-based models can be used in the clinical workflow for fully automated planning for simple sites like prostate, but for more complicated sites, there may yet be some hurdles to overcome yet. They can be completely robust models that take days to compute making them completely unfeasible, or they can be simplified to improve generalizability by trading the capability of handling a wide array of niche scenarios in which a human planner would be better fit to tackle. Despite this, there can be a lot to be gained from investigating the implicit knowledge of these models. Many models are built around simple, logical principles and utilize the experience of previous high-quality plans, so a user who lacks experience in planning can pick up on an important foundation and progress to a level of clinical reliability if the bridge between model and human is established. Properly extracting this information can provide anyone within the planning workflow an avenue to deeper understanding of its intricacies and what to look for in evaluation. The thought is to make a human centered—AI system to exploit the strengths from both ends and train competent planners.

It should be understood that systems and methods disclosed herein may be suitably applied to other applications for training apart from training for radiation therapy planning. Other training systems can benefit from being refined in efficiency to serve a purpose of quickly training those who utilize it. While long training and arduous hours of work can certainly create competent, professional planners, a degree of quickness benefits the greater population for training in cutting cost and getting helping planners working well become more proficient in the clinic sooner. Of course, there are aspects of planning that can only be obtained by years of nuanced planning, but plan quality is not always shown to be better in those who have more experience. Some planners with planning experience may encounter a bottle-neck in improving their versatility in planning various scenarios, due to the lack of understanding the underlying subtlety which can be readily provided and instructed by the knowledge-based model. In addition, training a planner to a high level in a traditional mentor-tutor fashion can be expensive in time and resources, and sometime the limited training resources are dispatched to more entry level learners. A person can quickly learn how to plan well if the teaching is well thought out and provides the base for the person to build their own intuition.

Also, it is noted that use of knowledge-based models within a clinical scenario for treatment planning can sometime be adopted slowly. There are a few reasons for this, and one of which is lack of guidance of how to properly utilize them. Most models, at this point, are best at supplementing the strategies of human planning and serve as either “jumping off points” or a place of reference to help make a decision. A learning system that introduces the benefit of these models can aid in tearing down the notion of them being entirely a black box. This system can be utilized as an introduction to the simple principles the models are based on for an understanding of how they work and how to best utilize them. This system can be a catalyst to bring more models into routine clinical work. One study that was implemented looks at taking advantage of two knowledge-based models and molding an intelligent learning system to facilitate efficient and quality learning of lung IMRT treatment planning with comparative scoring criteria to help establish intuition to trainees with no previous planning experience.

Systems disclosed herein can have an end goal use of being an entirely self-sufficient training system and move into the space of being a constraint-based intelligent tutor. Constraint-based intelligent tutors have a variety of explicit definitions depending on the field of interest being used, but here it entails a problem that does not have an explicit solution or path for a user to follow. Instead, it is comprised of defined constraints, in the form of the scoring system, and an end goal, which is simply for the user to optimize the scoring system to obtain the highest score possible. The user may need to forge his or her own path from the information that is directed to them, and two people can take entirely different strategies and arrive at good solutions. The systems disclosed herein also echo clinical practice which evaluates the eventual plan presented for physician to review regardless of path leading to it. This is a proof of concept study to show that there is valuable information to be gained from the models and they can be effectively and efficiently used in training new planners and give them the ability to utilize these models in making quality plans.

In some examples, a new trainee may be introduced to the treatment planning system with functionality they might not be familiar with as they had no prior experience. The trainee may be provided with the scoring metrics and asked to plan an intermediate-to-hard difficulty plan without any intervention from the trainer, knowledge-based models, or user interface. The difficulty was determined by looking at the prescription, tumor size, complexity of shape, and proximity to OARs. The trainee was instructed they have the choice between a 6 and 10 MV beam and can have no more than 11 beams to align with current clinical practice.

After the benchmark of intermediate-difficult rating with a target volume of 762.8 cm³ and a prescription of 62 Gy, the system involved 5 additional patients: two easier cases with an average target volume of 113.8 cm³ and prescriptions of 40 Gy, one intermediate case with a target volume of 453.0 cm³ and a prescription of 60 Gy, and two difficult cases with an average target volume of 845.7 cm³ and prescriptions of 60 Gy. A user interface script that provides a quick comparison of two plans with scores, DVHs, predicted DVHs, and objective constraints can be supplied for use for direct training.

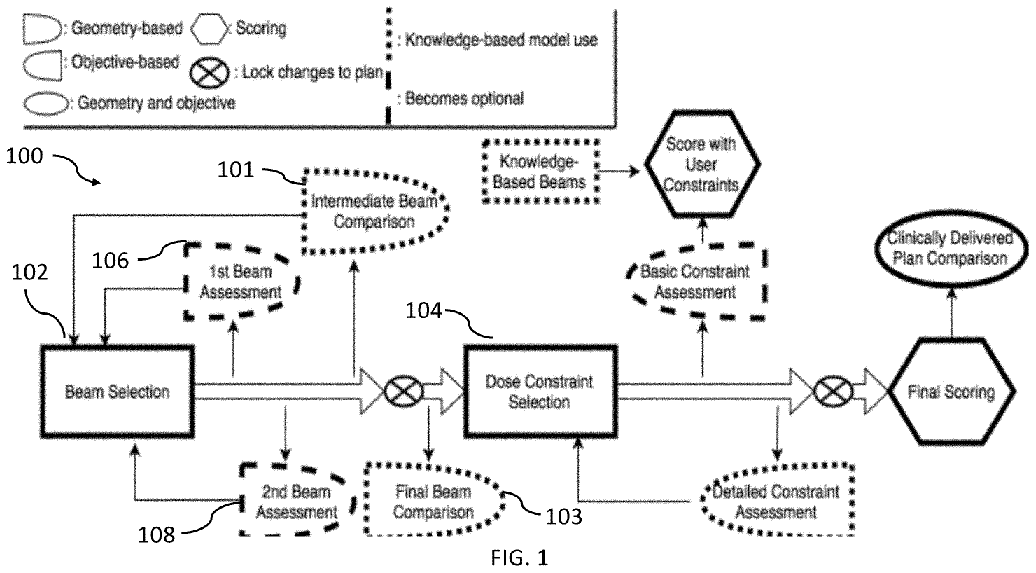

FIG. 1 illustrates a block diagram of a system 100 for radiation treatment planning based on knowledge data and trainee input of planning actions in accordance with embodiments of the present disclosure. Referring to FIG. 1, much of the interaction is on the basis of the trainee attempting to explain differences between their plan and a comparison plan and the resulting dosimetric implications of those differences. The comparison plan can change based on the trainee's progression within the system. The comparison plan can start as a plan that has inputted different number of beams from the beam model. This can start as seven beams for an intermediate comparison 101 and can end with 11 beams for the final comparison 103. Block 102 represents the step of beam selection, and block 104 represents dose constraint selection. The trainee can evaluate whether he or she wants to move in the direction of the model or continue with her or his own direction. In the final geometric comparison 103, the trainee can make an assessment on what the dosimetric differences in her or his plan versus the comparison is based on geometry. Subsequent to the beam setting being determined, the trainee can create initial optimization constraints and the constraints can be copied into the knowledge-based beam plan and a dosimetric comparison can be conducted to check whether the results aligned with the trainee's expectation, thus building a forward intuition on beam choice implications. Following this, the DVH model is imported and the trainee is able to compare their end DVHs and dose objectives to where the DVH model predicts the trainee is capable of achieving and the trainee can make changes based on what she or he takes away from what the model claims is obtainable. Subsequent to the changes being made, the plan can be scored, and the final comparison plan is the clinically delivered plan. The trainee can work backward by looking at the scoring and DVHs of the clinical plan and can make a judgement on how she or he thinks the clinically delivered plan was set up. This is to further ingrain in the trainee a backwards intuition for the metrics related to certain collective beam arrangements.

In accordance with embodiments, there can be two different points in training where no more changes may be made to that portion of the plan, right before making the final comparison the beam model and the iteration after making changes from what the trainee sees is expected from the DVH model. This can serve to cut back on constant iteration for small improvements and force more careful thought in the trainee's initial attempts. Although, it may be realized from a trial trainee that the more difficult plans may need more iteration, so the pipeline for the last three plans can give opportunity to change the beam selection after the dosimetric comparison between the trainee's plan and the beam model plan.

With continuing reference to FIG. 1, the figure shows steps that may be optional and that primarily used with trainees that have little to no knowledge of treatment planning, particularly those to whom this intelligent learning system is currently targeted towards. The first beam assessment (indicated by block 106) can be a review session with the trainer module to understand, if he or she were to make a plan with only a single beam, which direction would be best, and this tries to get the trainee to think of how each individual beam can contribute. The second beam assessment (indicated by block 108) can be used to make an optimal plan the best if only two beams are used, and this starts to instill how the beams may be interacting with one another (i.e., the second absolute best beam is not necessarily the best beam to work with the first). Lastly, the basic constraint assessment is a simple check the trainee has an objective for all the relevant structures and two for the target.

Following the five plans, the trainee can return to the same patient she or he did a baseline on and plan it again to see if there was any significant improvement in their planning ability. This can be done without any intervention from the trainer, knowledge-based models, or user interface.

In accordance with embodiments, the beam angle selection model operates on a simple efficiency index that tries to maximize the dose delivered to a PTV and minimize the dose delivered to OARs based on a number of learned weighting factors, and it can induce a forced separation between good quality beams in the efficiency index to not lose out on all semblance of conformality. For the purposes of simplicity of introduction to new planners, all beams may be restricted to co-planar. Users have the freedom to select the number of beams needed.

In accordance with embodiments, a DVH prediction model may utilize principal component analysis of a number of anatomical features: DTH principal components, OAR volume, PTV volume, OAR-PTV overlapping volume, and out-of-field OAR volume. The model was trained with 74 lung cases with a variety of tumor sizes and locations, and it predicts DVHs for: cord, cord+3 mm, lungs, heart, and esophagus. As the model is dependent on the out-of-field OAR volume, it is inherently dependent on the beam selection; the predictions were made based on the clinically delivered plan's beam selection, and an assumption was made that the trainee's beams were close enough to the beams that the clinically delivered plan was built upon. While that assumption might not always hold true, it is typically clear that when the trainee's beams differ by a large amount it results in an inferior quite poor plan.

In accordance with embodiments, a scoring system as disclosed herein can facilitate communication of comparison between different plans and the metrics with their respective max point value and is shown in table 1; these systems can be created in many ways, but this one was created with the initial logic of rewarding dosimetric endpoints that are clinically relevant. The conformality index (CI) can be used to limit the isodose volume and can be represented as the following equation:

$${CI}_{x \%} = \frac{{V}_{x \%}}{{V}_{PTV}} \qquad ( 1 )$$

The conformation number (CN) or Paddick conformity index follows similar logic to CI but focuses on the portion of that isodose volume within the target as follows:

$${CN}_{x \%} = \frac{{( {V}_{x \%}^{PTV} )}^{2}}{{V}_{PTV} \times {V}_{x \%}} \qquad ( 2 )$$

Within a scoring system as disclosed herein, knowing the metrics and priorities can provide some information on how to pursue a problem at hand. Unfortunately, this does not always hold true, so the scoring can be adjusted to hold a second function of having “guiding metrics” to lead towards clinically feasible plans. An example of this is V5Gy versus V20Gy of the lung. Physicians may more priority to V20Gy as an indicator of pneumonitis, but the nature of V5Gy being more sensitive to careful beam selection can mean a better guidance to how a person selects teir beams. V20Gy can be forced in an optimization without causing other metrics to be entirely infeasible, but the plan may not necessarily be as good as it would have with more meaningful beam selection. The guiding component is to weigh the priority of V5Gy higher, so the trainee puts priority on careful beam selection without producing a degenerate plan.

Table 1 below shows example metrics chosen to be part of a scoring system and their respective maximum point value in accordance with embodiments.

| | | | | | | | | | |
|---|---|---|---|---|---|---|---|---|---|
| Target | Max | Lung | Max | Heart | Max | Esophagus | Max | Spinal Cord | Max |

| | | | | | | | | |
|---|---|---|---|---|---|---|---|---|
| PTV D98% | 21 | Max dose | 5 | Max dose | 5 | Max dose | 7 | Cord max dose |
| PTV | 10 | Mean dose | 5 | Mean dose | 7 | Mean dose | 5 | Cord + 3 mm |
| min dose | | | | | | | | max dose |
| GTV | 10 | V20Gy | 10 | V30Gy | 5 | | | |
| min dose | | | | | | | | |
| GTV | 12 | V5Gy | 15 | V40Gy | 5 | | | |
| min dose | | | | | | | | |
| CN 95% | 12 | | | | | | | |
| Location of | 10 | | | | | | | |
| max dose | | | | | | | | |

Even with careful thought, an overall score within any of these systems are not necessarily going to be indicative of what is better in a real clinical scenario. To combat this, a physician specialized in lung service evaluated the plans by which the trainee planned as a benchmark and the plan following the training system. Two plan pair from each trainee was compared against one another, and the final plan was compared against the clinically delivered plan. The comparison was based on a simplified 5-point model of significantly worse (1), moderately worse (2), comparable (3), moderately better (4), significantly better (5). The physician also evaluated the trainee's final plan on whether she or he would approve it for treatment.

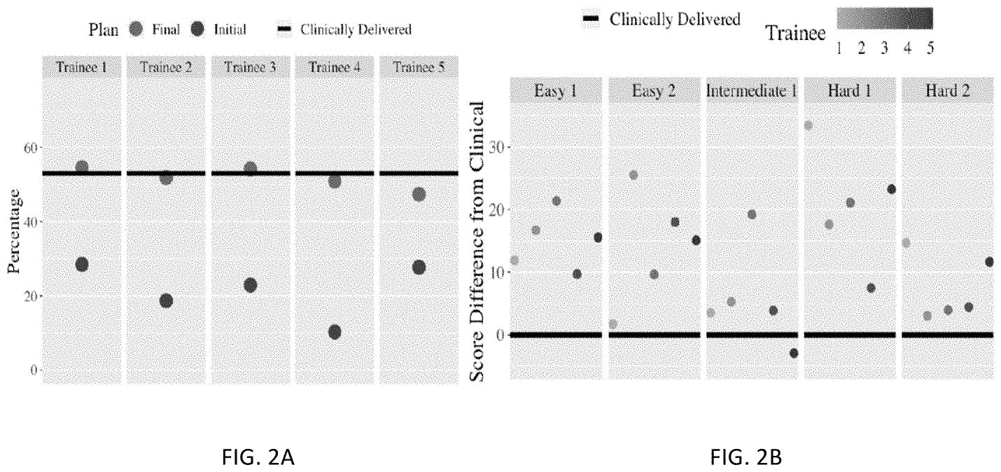

In experiments, five trainees went through the system and their scores are shown in FIGS. 2A and 2B, which are graphs showing trainee scores for a benchmark plan and five plans according to the present disclosure, respectively. Particularly, FIG. 2A shows trainee scores for a benchmark plan and their return compared to the clinically-delivered plan. FIG. 2B shows trainee scores for five plans within an intelligent learning system compared to a respective clinically delivered plan. For the five plans within the training system as shown in FIG. 2B, every trainee obtained a score that was greater than that of the clinically delivered plan with the exception of case 3 for trainee 5 and there was an overall average of 24.5% improvement over the respective clinically delivered plans. In terms of the trainee's improvement, their return was unanimously improved from their baseline and all received a score much closer to that of the clinically delivered plan. Trainee 1 and 3 received a score that was slightly above as the clinically delivered while, with an average of 54.6%, while the other three were marginally lower with an average of 50.8% compared to the clinically delivered of 54.1%.

Embodiments of the present disclosure demonstrate the feasibility that knowledge models can be used as effective teaching aids to train individuals to develop IMRT plans and in the process help human planners build confidence in utilizing the KBP models in a clinical setting. According to embodiments, a training program is provided that empowers trainers to effectively teach knowledge based IMRT planning aided by a host of training cases and a novel tutoring system that consists of a front-end visualization module, which is powered by training models that include knowledge models and a scoring system. The visualization module in the tutoring system provides the vital interactive workspace for the trainee and trainer, while the knowledge models and scoring system provide back-end knowledge support. This tutoring system may include a beam angle prediction model and a dose-volume histogram (DVH) prediction model as knowledge models. The scoring system includes physician chosen criteria for clinical plan evaluation as well as specially designed criteria for learning guidance. In an example implementation, a training program includes a total of six lung/mediastinum IMRT patient: one benchmark case and five training cases. Each case includes images, structures, and a clinically delivered plan, which are de-identified for learning. The benchmark case is used to show skill development from the training program. A plan for this benchmark case can be completed by each trainee entirely independently pre- and post-training. The five training cases cover a wide spectrum of complexity from easy (2), intermediate (1) to hard (2) in lung IMRT planning. To assess the tutoring system and the training program as a whole, five trainees with adequate medical physics course work but minimal clinical treatment planning practice went through the training program with the help of one trainer. Plans designed by the trainees were evaluated by both the scoring system and a radiation oncologist to quantify planning quality improvement and relative effectiveness against the clinically delivered plans.

For the benchmark case, trainees scored an average of 22.7% of the total max points pre-training and improved to an average of 51.9% post-training. In comparison, the clinical plans score an average of 54.1% of the total max points. Two of the five trainees' post-training plans on the benchmark case were rated as comparable to the clinically delivered plans by the physician and all five were noticeably improved by the physician's standards. The total training time for each trainee ranged between 9 and 12 hours. This first attempt at a knowledge model-based training program brought unexperienced planners to a level close to experienced planners in fewer than 2 days of time. It is believed that this tutoring system aided by knowledge models can serve as an important component of an AI ecosystem that can enable clinical practitioners to effectively and confidently use KBP in radiation treatment.

In embodiments, knowledge models collect and extract important patterns and knowledge from high quality clinical plans and utilize them to predict clinically optimal solutions for new cases. For treatment planning, this may come in the form of selected beam angles, optimized collimator settings, predicted achievable dose-volume histogram (DVH) endpoints for inverse optimization, and combined multiple parameter predictions for a fully automated treatment planning process (1-14). Knowledge models have been successfully used in the clinical workflow for fully automated planning for some simpler cancer sites like prostate, but for more complicated sites, there may yet be some hurdles to overcome. Due to limitation of training samples and other factors, they are often simplified to improve generalizability by trading the capability of handling a wide array of niche scenarios in which a human planner would be better fit to tackle. Despite this, there is a lot to be gained from investigating the implicit knowledge of these models. The simple, logical principles that most of these models are built upon can not only start a foundation for less experienced users to progress towards clinical reliability but also bridge the gap between human and model knowledge in what to look for in evaluation and identification of planning intricacies. The thought is to make a human-centered artificial intelligence system to exploit the strengths from both ends and efficiently train competent planners.

According to embodiments, a training program system as provided herein lays the foundation for an entirely self-sufficient training system that will be designed as a constraint-based intelligent tutoring system (ITS). The constraint-based approach supports the type of learning problem that does not have an explicit solution or path for a user to follow as is the case of IMRT planning. The constraints are defined in the form of the scoring system, and the end goal is for the user to learn the planning actions that optimize the scoring system to obtain the highest score possible. In this constraint-based framework, the user can forge her or his own path from the information that is directed to them, and two people can take entirely different strategies and arrive at good solutions.

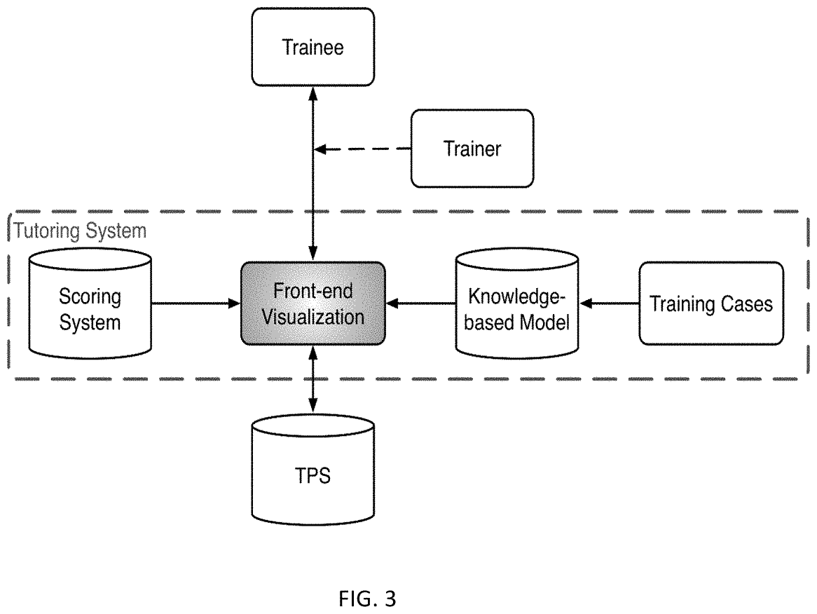

FIG. 3 illustrates a block diagram of an example training system in accordance with embodiments of the present disclosure. The overall training program design is shown in FIG. 3. Referring to FIG. 3, the system includes tutoring module and a host of training cases. The tutoring module can bring together the trainee, trainer, and the TPS. A trainer can be optional for assisting the interaction between the trainee and the tutoring module. The tutoring module can be powered by a scoring system and a set of knowledge models. At the core of the training program is the tutoring system which consists of a front-end visualization module powered by KBP models and a plan scoring system. The visualization module provides the vital interactive workspace for the trainee and trainer, while the KBP models and scoring system provides back-end knowledge support. The KBP models currently include a beam bouquet prediction model and a DVH prediction model, while the scoring system consists of physician chosen criteria for clinical plan evaluation as well as specially designed criteria for learning guidance. Further, the tutoring system works in concert with the clinical treatment planning system (TPS) as trainees learn to generate clinically plans in a realistic clinical planning environment.

The current training program has selected a total of six lung/mediastinum IMRT patient cases: one benchmark case and five training cases. Each case is composed of clinical images, structures, and a delivered plan which were de-identified before incorporated into the training program. The benchmark case is used to track skill development. The five training cases cover the complexity from easy (2), intermediate (1) to hard (2) in lung IMRT planning. The difficulty level is determined by an experienced planner who evaluated the prescription, tumor size, complexity of shape, and proximity to OARs. The benchmark case, considered “intermediate-to-hard”, has a target volume of 762.8 cc and a prescription of 62 Gy; two “easy” training cases have an average target volume of 113.8 cc and prescription of 40 Gy; the “intermediate” training case has a target volume of 453.0 cc and a prescription of 60 Gy; two “hard” training cases have an average target volume of 845.7 cc and prescriptions of 60 Gy.

Before training begins, each trainee can undergo a benchmarking process first. In this process, the trainee is introduced to the treatment planning system with functionality that he or she may not be familiar with as the trainee has no prior experience. They are provided with the scoring metrics and asked to plan the benchmark case without any intervention from the trainer or the tutoring system (referred to as the baseline plan). The trainee is instructed that they have the choice of 6 or 10 MV beams and could have no more than 11 beams to align with current clinical practice.

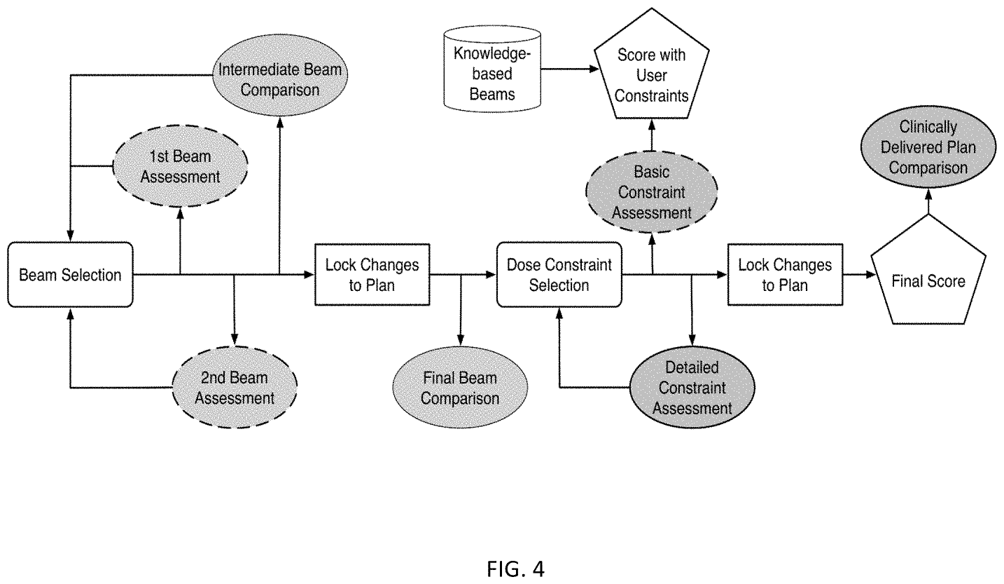

FIG. 4 illustrates a flow diagram showing an example training workflow (solid lines) for learning to plan a training case in accordance with embodiments of the present disclosure. The cases are selected sequentially from the easy cases to the difficult cases. For each case, a trainee can undergo two phases of training: the beam selection phase and the fluence map optimization phase. In both phases, each training episode involves three main steps: (1) the trainee makes a decision (or takes an action); (2) the training program generates a plan corresponding to the decision and displays relevant dose metrics; (3) the training program then generates a comparison plan according to predictions from knowledge models and displays the same set of relevant dose metrics for comparison. The majority of interaction centers around the process with which the trainee learns to explain the differences between their plan and the comparison plan, as well as the resulting dosimetric implications of those differences.

During the beam selection phase, the trainee can choose the number of beams (seven to eleven) and the direction of beams. The comparison plans are those generated with beams determined by the knowledge-based beam selection model. Both trainee plans and comparison plans are created by an automatic KBP algorithm. The trainee determines whether they prefer to move along the direction of model prediction or continue with their own direction. At the end of this phase, the final beam comparison provides an assessment of the expected dosimetric differences contributed by trainee's beam design.

When the optimization training phase begins, the trainee creates the initial optimization objectives and finishes the planning process. In parallel, a comparison plan is generated with the KBP beam setting using trainee's dose-volume constraints. And the dosimetric comparison between plans allow the trainee to appreciate whether the results aligned with their expectations during aforementioned assessment, which builds a forward intuition on beam choice implications.

Following this, the KBP DVH model can be imported and the trainee is able to compare their plan's DVHs and dose objectives to where the DVH model predicts they should be able to achieve. The trainee can subsequently make changes based on what they see is obtainable. Subsequent to the changes being made, the plan is scored, and the final comparison plan is the clinically delivered plan. The trainee works backwards by looking at the scoring and DVH of the clinical plan and ponders on how the clinically delivered plan might have been achieved. This is to further ingrain a backwards intuition for the metrics related to certain collective beam arrangements.

As shown in FIG. 4, the training workflow can include a few steps (dashed boxes) that are designed for people with little to no knowledge of treatment planning; these steps are optional when trainees are at more advanced stages during the training process. The first beam assessment is an initial guidance with the trainer about the best beam direction to select if they were to make a plan with only a single beam. This step encourages the trainee to think about how each individual beam will contribute to the dose distribution. The second beam assessment helps the trainee make an optimal plan when only two beams are used, and this starts to instill the knowledge about how multiple beams interact with one another (i.e., the second best beam is not necessarily the best beam to work with the first). Lastly, the “basic constraint assessment” step is a simple check to ensure that the trainee has at least one objective for all the relevant structures and two for the target.

The current training program takes the trainee through the workflow described in FIG. 4 five times, one for each training case, in increasing order of difficulty. After completing all five cases, the trainee returns to the benchmark case and creates a new plan entirely on his or her own without any intervention from the trainer, knowledge models, or the tutoring system. This post-training plan in comparison to the baseline plan on the same case provides an objective way to assess if there is any significant improvement in their planning ability.

As introduced in the previous section, the current tutoring system includes three major components: visualization module for user interaction, knowledge models for planning guidance, and a scoring system for plan assessment. The visualization module can be implemented as a script an application programming interface (API). In the following description, additional details of the knowledge models and the scoring system are provided.

The beam model predicts the best beam configuration for each new case, including the number of beams and the angle of the beams. It operates on a novel beam efficiency index that tries to maximize the dose delivered to a PTV and minimize the dose delivered to OARs based on a number of weighting factors, and it introduces a forced separation between good quality beams to not lose out on overall balance of conformality. The weighting factors and other parameters of the beam model are learned from a set of high quality prior clinical cases. For the purposes of simplicity of introduction to new planners, all beams in the current training program are restricted to co-planar beams.

The DVH prediction model can estimate the best achievable DVH of the OARs based on a number of anatomical features: distance-to-target histogram (DTH) principal components, OAR volume, PTV volume, OAR-PTV overlapping volume, and out-of-field OAR volume. The model is trained with a set of prior lung cases with a variety of tumor sizes and locations. For this study, the model predicts DVHs that are useful for the trainees during the learning and planning, they include: cord, cord+3 mm, lungs, heart, and esophagus.

A plan scoring system can be used to help trainees understand the quality of different plans from the choices of beams and DVH parameters. Therefore, the scoring system incorporates both physician's clinical evaluation criteria and planning knowledge. The metrics with their respective max point values are shown in Table 2. As noted, since each case has its own unique anatomy and complexity, the best achievable points of a plan is always less than the total max points, and the more difficult the case, the least best achievable points for a plan. The best achievable points of each plan is not normalized so the trainees are encouraged to rely on the actual planning knowledge to “do the best”, rather than to get “100 percent score” or gaming the system. Note that even clinically delivered plans may not be perfect in all categories, and therefore, may not achieve the highest possible scores.

TABLE 2
Metrics chosen to be a part of the scoring system and their respective maximum point value.

| Target | Max | Lung | Max | Heart | Max | Esophagus | Max | Spinal Cord | Max |
|---|---|---|---|---|---|---|---|---|---|
| PTV D98% | 21 | Max dose | 5 | Max dose | 5 | Max dose | 7 | Cord max dose | 10 |
| PTV | 10 | Mean dose | 5 | Mean dose | 7 | Mean dose | 5 | Cord + 3 mm | 10 |
| min dose | | | | | | | | max dose | |
| GTV | 10 | V20Gy | 10 | V30Gy | 5 | | | | |
| min dose | | | | | | | | | |
| CN 95% | 12 | V5Gy | 15 | V40Gy | 5 | | | | |
| CI 50% | 12 | | | | | | | | |
| Location of | 10 | | | | | | | | |
| max dose | | | | | | | | | |

An effective scoring system can be created in one of a suitable manner described herein, the current system starts with the logic of rewarding dosimetric endpoints that are clinically relevant as explained in various RTOG reports, other clinical considerations and planning competitions. The conformality index (CI) aims to limit the isodose volume. The conformation number (CN) or Paddick conformity index follows similar logic to CI but focuses on the portion of that isodose volume within the target.

To assess the effectiveness of the training program, five trainees with adequate medical physics course work but minimal clinical treatment planning practice went through the entire training program. For all five trainees, the baseline and post-training plans of the benchmark case were scored and analyzed for evidence of learning. Furthermore, the post-training plans of all the training cases as well as all the clinically delivered plans were also scored and analyzed for trainee performance and potential knowledge gaps.

Moreover, to assess how the overall scores given by the scoring system closely reflect true plan quality in a real clinical scenario, a physician specialized in lung service evaluated the plans to provide an expert opinion on their clinical quality. For each trainee, the post-training plan of the benchmark case was first compared with the baseline plan of the same case and then against the clinically delivered plan by the physician. Each comparison was categorized on a simplified 5-point scale of (1) significantly worse, (2) moderately worse, (3) comparable, (4) moderately better, (5) significantly better. The physician also evaluated the trainee's post-training plans on whether they could be approved for clinical delivery.

Table 3 below shows the physician evaluation of the trainee's post-training plans as compared to the benchmark plans and the clinically delivered plans. Two plans designed by trainees #1 and #3 that scored slightly better than the clinical plan per scoring system were deemed as comparable to the clinical plan by the physician. The other plans were rated as marginally worse. All trainee's post-training plans were rated moderately better than the initial benchmark plans. Only one of the trainee's plan was deemed entirely ready to be delivered based on the physician's discretion.

TABLE 3
Physician evaluation of trainee post-training plan
on 5-point scale (significantly worse to significantly
better) and clinical feasibility rating

| | Comparison to | Comparison to | Clinically |
|---|---|---|---|
| Trainee # | Clinical | Benchmark | Feasible |
| 1 | Comparable | Moderately | No |
| | | Better | |
| 2 | Moderately | Moderately | No |
| | Worse | Better | |
| 3 | Comparable | Moderately | Yes |
| | | Better | |
| 4 | Moderately | Moderately | No |
| | Worse | Better | |
| 5 | Moderately | Moderately | No |
| | Worse | Better | |

Radiation treatment planning is fundamentally the key procedural skill that is used by radiation oncologists to deliver curative or palliative treatment for adults and children with cancer. Radiation is used to treat approximately 50% of patients diagnosed with cancer, ½ for curative intent and ½ to palliate symptoms. The process of radiation treatment planning starts with a CT scan that is done in the position that the patient will receive radiation. Physicians then identify targets to focus high radiation doses to and normal tissues/organs to avoid exposure to radiation. The short and long term side effects a patient may experience is directly associated with the amount of radiation an organ receives, and there is always a balance between delivery of enough radiation to control tumors, while minimizing dose to surrounding organs. Therefore, it is critical that during the course of training, radiation oncology residents become skilled in the methods to design optimal radiation plans.

In current practice, each resident's development of radiation planning knowledge entirely relies on one-to-one interaction with their assigned attending physician, who reviews each patient's radiation plan on the 3D CT/MRI image sets, and explains the “knowledge” embedded in the decision making of revising and approving a radiation plan, based on prior experience, clinical evidence, biological modeling, and guidelines. Such learning is direct but restrictive, requiring synchronized schedule of the resident and the attending, which is becoming much more difficult to coordinate given increasing clinical demands of patients, payors, and the electronic medical record. With an ever more dynamic healthcare system and clinical care environment, it is believed that building a parallel asynchronized learning system can complement the traditional apprentice model of medical teaching.

To address this need, an intelligent learning system has been developed based on modeling radiation planning knowledge. The intelligent learning system can detect the deficiency of a particular knowledge unit, and dynamically compose the presentation of this knowledge unit, assemble patient cases for learning and practicing, then provide another test module to confirm the mastery of the knowledge unit. This system can take full advantage of the latest technologies including artificial intelligence and cloud computing, with our expertise of implementing such technologies in radiation oncology field.

Like many other medicine specialties, the radiation planning knowledge domain is complex and multi-dimensional, they are not covered by existing intelligent learning research and products in both content and design principles. Such knowledge domain requires extensive integration of clinical expertise from physician and physicist experts, and radiation physics, biology and medical principles. As a preliminary work, an intelligent learning program has been developed that incorporates two knowledge models as a learning aid for lung cancer radiation planning.

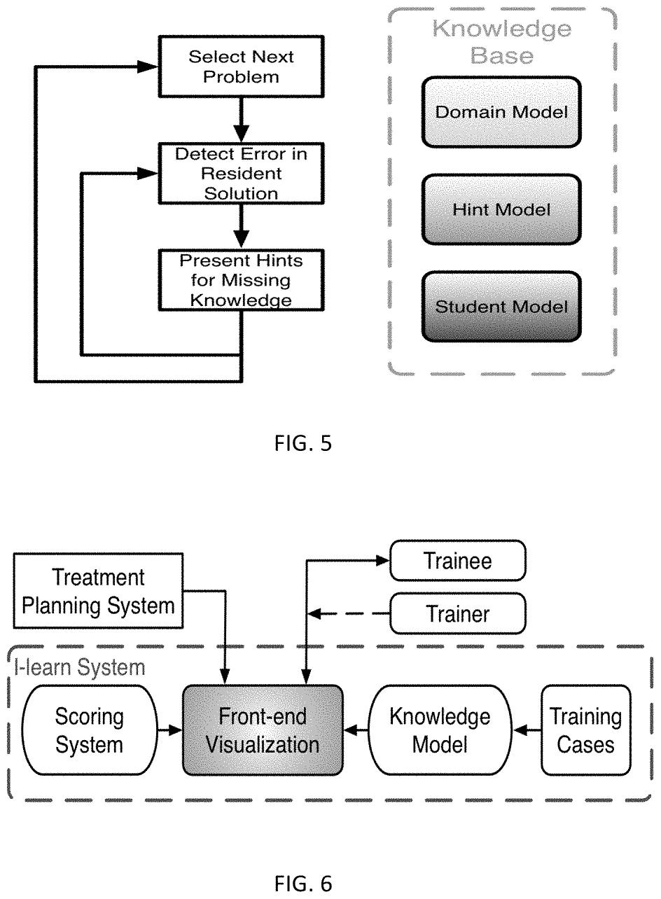

In accordance with embodiments, FIG. 5 illustrates a flow diagram of an intelligent learning method. Referring to FIG. 5, the intelligent learning system can work in two nested loops. The intelligent learning system can detect the deficiency of a particular knowledge unit, and dynamically generate presentation of this knowledge unit (inner loop), assemble patient cases for learning and practicing, and subsequently provide another test module to confirm the mastery of the knowledge unit (outer loop). In the core is a Knowledge Base that acts as the brain of the system, which can be divided into three knowledge model groups: a domain model, a hint model, and a student model. The domain model represents the collection of knowledge units and includes a set of practice cases. The hint model represents knowledge about mistakes that trainees may make and the learning materials that are designed to help trainees learn from the mistakes. The student model represents knowledge about the trainee's understanding of domain knowledge based on history of trainee actions and performances. With the help of these models, the intelligent learning system can detect gaps in a trainee's knowledge about the domain and deliver appropriate learning cases and materials to help the trainee fill the gaps.

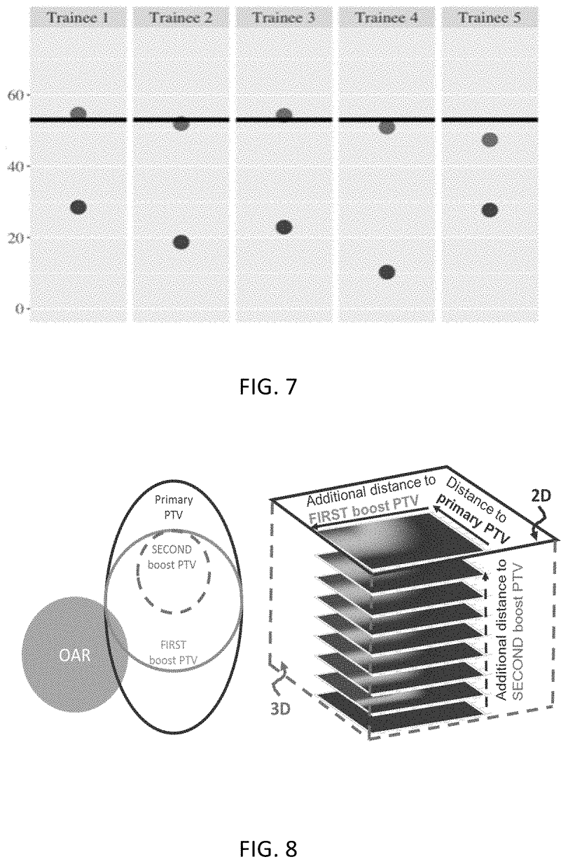

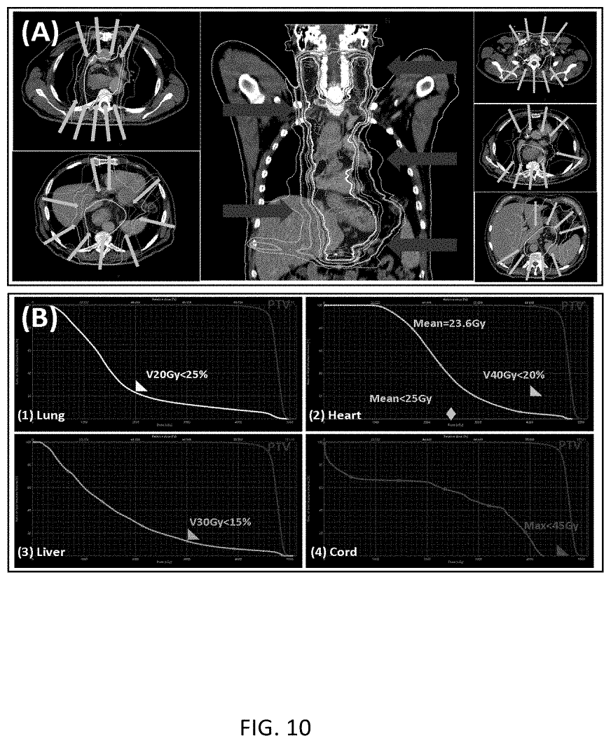

Following the intelligent learning system foundations outlined in FIG. 5, FIG. 6 illustrates a block diagram of an intelligent learning system in accordance with embodiments of the present disclosure. Referring to FIG. 6, the three major components for radiation planning workflow: visualization module for user interaction, two knowledge models for treatment planning guidance, and a scoring system for assessment. The visualization module is the centerpiece for user interaction during learning. Furthermore, there are two main types of visualization. The first type displays various knowledge components and its associated anatomies and cases (see FIGS. 8 and 10(A)). The second type displays various plots that indicate the status and comparisons of the learning progress (see FIGS. 7, 9, and 10(B)). FIG. 7 illustrates a graph depicting knowledge improvements after use of the intelligent learning program (the upper dots in each column represents knowledge after use of the program, the lower dots in each column represents knowledge before use of the program, and the horizontal line in each column represent clinical expert knowledge).

FIG. 8 are images that illustrate gDTH, which is a 2D feature map when there is only 1 primary and 1 boost PTV volume, and becomes high dimensional with additional volumes.

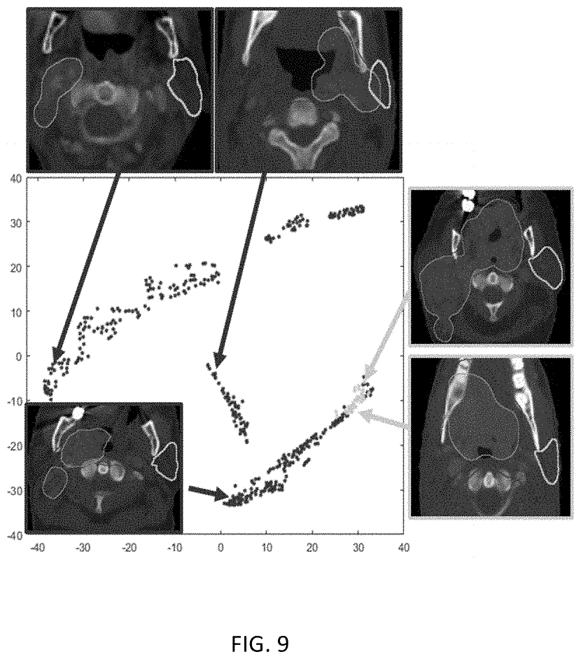

FIG. 9 illustrates a graph and images showing interpretation and visualization of gDTH for left parotid. Dots and boxes represent two similar cases as seen in the axial CT, while other boxes represent dissimilar cases.

FIG. 10 illustrates different images showing beam configuration and a dose-volume histogram. Particularly, the upper portion (designated (A)) shows that beam configuration varies longitudinally for the example esophagus case. The lower portion (designated (B)) shows the dose-volume histogram of the organs with physician prescribed dose objectives designated by a triangle (upper limit) and diamond (mean dose limit) for lung, heart, and spinal cord.

A collection of models has been developed that analyzes patient's unique tumor anatomy and predicts the best knowledge about the tumor coverage and organ exposure range. This pilot study tested two of these knowledge models for lung cancer radiation planning. The first is a radiation beam model which predicts the best bouquet for each patient, including the number of beams and the angles of the beams. The second knowledge model estimates the best achievable dose sparing of each normal organ based on a number of anatomical features. While the first knowledge unit focuses on choosing the organ sparing priorities and thresholds, the second devotes to present the interplay among multiple organ sparing considerations. For each lung cancer case, an experienced physician utilizes these two key knowledge units intuitively and collectively in his/her mind when designing and reviewing a radiation plan.

The scoring system can help trainees understand the quality of their radiation plans. The scores are carefully designed to reflect trainee's understanding of each knowledge components. Since each patient case has its own unique anatomy and complexity, the best achievable scores of a radiation plan is always less than the total max scores (representing ideal condition), and the more difficult the case, the less best achievable scores for a plan. Therefore, the best achievable scores of each patient case is not normalized such that the trainees are encouraged to rely on the actual planning knowledge to “do the best possible”, rather than to get “100 percent score” or gaming the system (as shown in FIG. 7).

As an initial trial, five trainees went through the learning program which took around 10 hours. Prior to the trial, all trainees have gone through classroom courses on radiation physics, anatomy, radiation biology and treatment planning. They also have received a basic practicum course to learn the essential operations of the treatment planning system. As shown in FIG. 7, all trainees improved their scores significantly from the baseline after going through the learning program and their scores were much closer to that of the clinical expert's level. In a second assessment, a physician evaluated the post-learning plans—2 of 5 plans were deemed as comparable to the clinical expert level and the other plans were rated as marginally worse.

To help physicians understand a knowledge unit, use of t-sne as the visualization and interpretation layer was explored, as shown in FIG. 9. The central plot is t-sne of 200 patient cases, where various clustered dots indicating cases of similar gDTHs of the left parotids, and other dots are less similar. Also plotted are the snapshots of patient's anatomy. This way, physicians can make the link between the gDTH concept and patient's anatomy—the format physicians are most familiar with and accustomed to digest and visualize their knowledge. In this example, physicians learned the left parotid exposure are similar for the two anatomies in the box, even though they have different tumor sizes and shapes.

Now referring to FIG. 10, a complex esophageal case is presented. The beam knowledge model covers multiple radiation planning knowledge components as the tumor extends from c-spine to abdomen. In the c-spine region, the beams (arrows in small windows) are evenly distributed around the tumor (knowledge unit: dose limit to spinal cord, dose homogeneity); however in the thorax region the beams have to avoid passing through lungs to minimize low dose regions in lungs (knowledge unit: lung low dose, dose conformity). As in the abdomen, the dose to the liver is significantly lower than any toxicity thresholds (knowledge unit: liver dose and toxicity, gDTH), demonstrating the as low as reasonably achievable (ALARA) concept of radiation planning.

Physicians and physicists can carefully identify and categories the knowledge units of each patient case; and design the corresponding content materials. Equal importantly, the trainees should not only understand the knowledge being overlaid/presented onto the current training case, but also be able to generalize such knowledge when similar scenarios calls for. For example, extensive mediastinum lung cases would call for the same heart-lung dose balance knowledge as this esophageal case. Therefore, assembling patient cases based on trainee's knowledge component needs is a highly effective way to configure domain knowledge model and ensure the trainee fully grasp the knowledge.

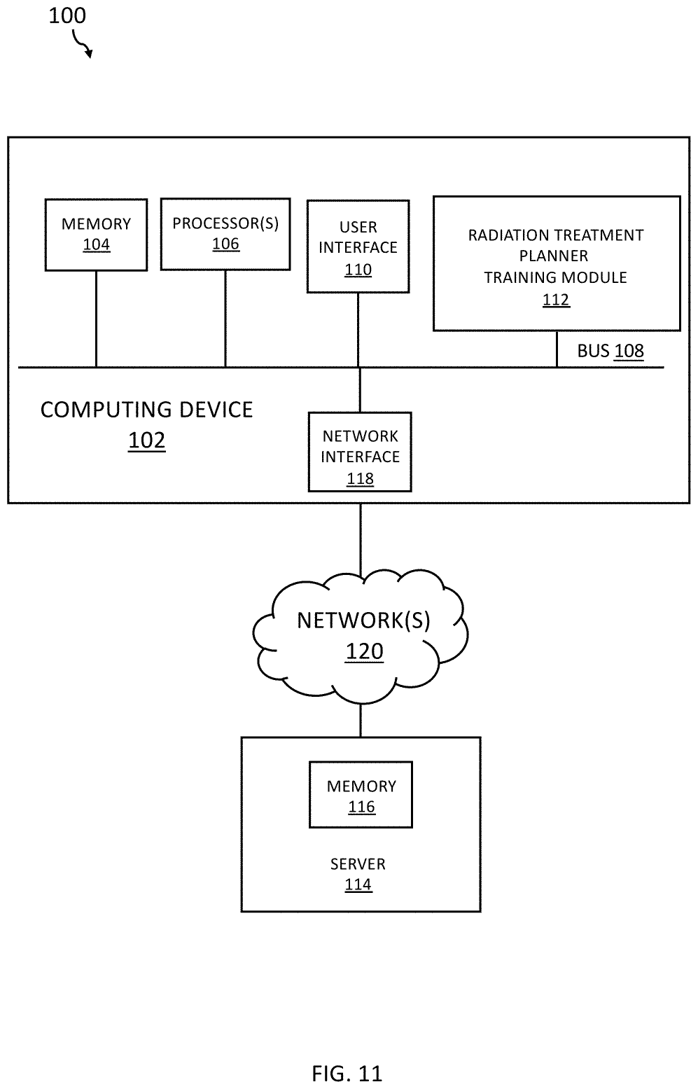

FIG. 11 illustrates a block diagram of an example system 100 for radiation treatment planner training based on knowledge data and trainee input of planning actions in accordance with embodiments of the present disclosure. It is noted that functionalities of the system 100 are described in this example as being implemented by a single computing device 102, although it should be appreciated that these functionalities may alternatively be implemented by one or more computing devices located locally together or remote from one another. The computing device 102 may include hardware, software, firmware, or combinations thereof for implementing the described functionalities. For example, the computing device 102 includes memory 104 and one or more processors 106 operatively connected by a bus 108. Further, the computing device 102 may include a user interface 110 (e.g., display, mouse, keyboard) for presentation of data and graphics to a user and for receipt of input from the user.

The computing device 102 includes a radiation treatment planner training module 112 is configured to receive case data including anatomical and/or geometric characterization data of a target volume and one or more organs at risk. The module 112 can also present, via the user interface 110, the case data to a trainee. Further, the module 112 can receive, via the user interface 110 from the trainee, input indicating an action to apply to the case. The input indicates one or more parameters for generating a treatment plan that applies radiation dose to the target volume and one or more parameters for constraining radiation to the one or more organs at risk. Further, the module 112 can apply training models to the action and the case data to generate analysis data of at least one parameter of the action. The module 112 can present, via the user interface 110, the analysis data of the at least one parameter of the action.

Knowledge and data as described herein can be accumulated and stored in any suitable manner such as in memory 104 of the computing device or remotely at a remote server's 114 memory 116. The module 112 may use a network interface 118 of the computing device 102 to access and retrieve, via one or more networks 120, the data stored in memory 116 of the server 114. Case data can include, but is not limited to, patient's medical image data, tumor target, and normal critical organ structure information. In other examples, the case data includes patient disease information, physiological condition information, and physician instruction including prescription. In other examples, case data includes patient disease information and other medical condition information. Anatomical and/or geometric characterization data can include data of a subject. The training models may be from within the system from multiple units. It is noted that the target volume can include total dose and daily fraction dose.

Analysis data can include, but is not limited to, radiation therapy principles about treating having the same or similar disease condition or other medical condition, anatomical data, and/or geometric characterization data as the subject. Further, analysis data can include radiation therapy principles including practice guidelines, historical radiation toxicity data, radiation biology data, radiation physics guidelines and theories about treating having the same or similar disease condition, other medical conditions, anatomical data of the tumor and normal organs, and/or geometric characterization data as the subject. In other examples, analysis data can include radiation therapy toxicity data about treating having the same or similar disease condition, anatomical data, and/or geometric characterization data as the subject. Further, the analysis data can include one of a beam angle selection and a dose-volume or dose constraint selection and a priority of that constraint based on knowledge models within the system and from one or more knowledge units.

Example input indicating action can include, but is not limited to one of a beam angle selection and a dose-volume selection for application to the target volume. In another example, input can include a dose-volume selection for application to one or more organ at risk. Further, the action can include one or more parameters about total dose, daily dose fraction, coverage criteria to the target volume.

Further, in an example, a target volume can include a part of the liver, lung, kidney, brain, neck, stomach, pancreas, spine, bladder, and/or rectum, or other body regions where tumor resides. Example organs at risk can include heart, lung, kidney, liver, brain, neck, stomach, pancreas, spine, bladder, and/or rectum, or other relevant radio-sensitive normal tissues and organs. Parameters can indicate, for example, a maximum radiation dosage to the one or more organs at risk as a whole or a certain portion of the volume. In another example, parameters can indicate a total radiation dose and fraction of one or more target volumes in total or partially with the dose constraint parameters of one or more organs at risk.

In accordance with embodiments, module 112 can receive, via the user interface 110 from the trainee, a request or a question regarding the analysis data. Further, the module 112 can present, via the user interface 110, the feedback for the trainee regarding the at least one parameter of the action.

In accordance with embodiments, the module 112 can receive case data comprising subject disease information, other medical condition information, and/or anatomical and/or geometric characterization data of a target volume and one or more organs at risk of a subject. Further, the module 112 can provide identification of one or more treatment plan analysis points for the subject. The module 112 can also provide knowledge data about treatment of the subject at the one or more treatment plan analysis points. The module can also present, via the user interface 110, the case data to a trainee and the identified one or more treatment plan analysis units, and questions regarding the units. Further, the module 112 can receive, via the user interface 110 from the trainee, response input to presentation of the one or more treatment plan analysis points. The module 112 can also generate analysis data of trainee feedback based on knowledge data and the response input in response to receipt of the response input. Further, the module 112 can presenting the analysis data via the user interface to the trainee in response to receipt of the response input.

Example case data can include other medical condition information, tumor target and normal critical organ structure information. Knowledge data can include practice guidelines, example patient cases, synthetic or simulated case/scenarios, and/or radiation physics principles. Trainee feedback can include a principle about treating having the same or similar condition, anatomical data, and/or geometric characterization data as the subject. Example principles includes, but are not limited to, specific practice guideline, radiation toxicity data, beam angle preferences, and/or organ sparing preferences. Further, trainee feedback can include one of a beam angle selection, a dose-volume selection, or a total dose and fraction selection based on knowledge data.

Example received input can include, but is not limited to, input indicating the action comprises receiving, via the user interface, one of a beam angle selection, a dose-volume selection, or a total dose and fraction selection for application to the target volume. The function of determining trainee feedback can be based on the one of a beam angle selection, a dose-volume selection, or a total dose and fraction selection. The trainee feedback can include visual feedback comprising a dose-volume histogram (DVH) curve and/or three-dimensional dose distributions of the subject. The response input can include selection of a point on the DVH curve. The trainee feedback comprises indication of another point on the DVH curve. Further, the trainee feedback comprises indication of normal tissue complication probability (NTCP).

In accordance with embodiments, the module 112 can presenting, via the user interface, the analysis data of the at least one parameter of the action. Further, the module can receive, via the user interface 110 from the trainee, request regarding the analysis data. The module 112 can also present, via the user interface 110, the most appropriate feedback for the trainee regarding the at least one parameter of the action.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A method comprising:
at at least one computing device:
receiving case data including patient disease information, historical medical information, anatomical and/or geometric characterization data of a target volume and one or more organs at risk;
presenting, via a user interface, the case data to a trainee;
receiving, via the user interface from the trainee, input indicating an action to apply to the case, the input indicating one or more parameters for generating a treatment plan that applies radiation dose to the target volume and one or more parameters for constraining radiation to the one or more organs at risk;
applying training models to the action and the case data to generate analysis data of at least one parameter of the action; and
presenting, via the user interface, the analysis data of the at least one parameter of the action.

2. The method of claim 1, wherein the case data comprises patient's disease information, historical medical information, medical image data, tumor target, normal critical organ structure information, and/or treatment objectives including total dose and fractions.

3. The method of claim 1, wherein the target volume comprises shape and volume of the gross tumor, clinical tumor volume, internal tumor volume with consideration of breathing and other motion, planning target volume, from one or multiple imaging modalities.

4. The method of claim 1, wherein the training models are from within the system from multiple units and include knowledge models for treatment planning, domain models for treatment principles, hint models for feedback, and student models for trainee evaluation.

5. The method of claim 1, wherein the anatomical and/or geometric characterization data comprises data of a subject.

6. The method of claim 1, wherein the analysis data comprises a radiation therapy principle about treating having the same or similar disease condition or other medical condition, anatomical data, and/or geometric characterization data as the subject.

7. The method of claim 1, wherein the analysis data comprises radiation therapy principles including practice guidelines, historical radiation toxicity data, relevant plan quality data, radiation biology data, radiation physics guidelines and theories about treating having the same or similar disease condition, other medical conditions, anatomical data of the tumor and normal organs, and/or geometric characterization data as the subject.

8. The method of claim 1, wherein the analysis data comprises a radiation therapy toxicity data and relevant plan quality data about treating having the same or similar disease condition, anatomical data, and/or geometric characterization data as the subject.

9. The method of claim 1, wherein the analysis data comprises one of selection total dose and fractions strategy to the one or more tumor target volumes and priorities based on training models within the system and from one or more knowledge units.

10. The method of claim 1, wherein receiving input indicating the action comprises receiving one of a beam energy and angle selection and a dose-volume selection for application to the target volume.

11. The method of claim 1, wherein receiving input indicating the action comprises receiving a dose-volume selection for application to one or more organ at risk and priority.

12. The method of claim 1, wherein receiving input indicating the action comprises receiving one or more parameters about total dose, daily dose fraction, coverage criteria to the target volume.

13. The method of claim 1, wherein the target volume comprises part of the liver, lung, kidney, brain, neck, stomach, pancreas, spine, bladder, and/or rectum, or other body regions where tumor resides.

14. The method of claim 1, wherein the one or more organs at risk comprises heart, lung, kidney, liver, brain, neck, stomach, pancreas, spine, bladder, and/or rectum, or other relevant radio-sensitive normal tissues and organs.

15. The method of claim 1, wherein the one or more parameters indicate a maximum radiation dosage to the one or more organs at risk as a whole or a certain portion of the volume.

16. The method of claim 1, wherein the one or more parameters indicate a total radiation dose and fraction of one or more target volumes in total or partially with the dose constraint parameters of one or more organs at risk.

17. The method of claim 1, wherein the one or more parameters indicate a parameter about a radiation beam, including energy, incident angles of static beam, start or stop angles of the rotation, beam size and shape.

18. The method of claim 1, further comprising:
receiving, via the user interface from the trainee, a request or a question regarding the analysis data; and
presenting, via the user interface, the feedback for the trainee regarding the at least one parameter of the action.

19. The method of claim 1, wherein the analysis data comprises missing knowledge of the trainee,
wherein the method further comprises:
presenting the missing knowledge; identification of missing knowledge and links of missing knowledge to one or more training models within the system
receiving input of action from the trainee; and
updating a plan based on the received input.

20. The method of claim 19, wherein presenting the missing knowledge comprises presenting an error of taking the action, an explanation of the missing knowledge, an example case presenting the missing knowledge, a simulation or synthetic phantom case presenting the missing knowledge, a list of references of published practice guidelines, and/or a corrective action.

21. The method of claim 19, wherein presenting the missing knowledge comprises presenting the missing knowledge via text, graphics, audio, and/or animation.

22. A method comprising:
at at least one computing device:
receiving case data comprising subject disease information, other medical condition information, and/or anatomical and/or geometric characterization data of a target volume and one or more organs at risk of a subject;
providing identification of one or more treatment plan analysis points for the subject;
providing knowledge data about treatment of the subject at the one or more treatment plan analysis points;
presenting, via a user interface, the case data to a trainee and the identified one or more treatment plan analysis units, and questions regarding the points;
receiving, via the user interface from the trainee, response input to presentation of the one or more treatment plan analysis points; and
in response to receipt of the response input:
generating analysis data of trainee feedback based on training models and the response input; and
presenting the analysis data via the user interface to the trainee.

23. The method of claim 22, wherein the case data comprises disease information, other medical information, tumor target and normal critical organ structure information.

24. The method of claim 22, wherein the knowledge data comprises practice guidelines, example patient cases, synthetic or simulated case/scenarios, and/or radiation physics and radiation biology principles.

25. The method of claim 22, wherein the trainee feedback comprises a principle about treating having the same or similar condition, anatomical data, and/or geometric characterization data as the subject.

26. The method of claim 25, wherein the principle comprises specific practice guideline, radiation toxicity data, beam energy and angle preferences, and/or organ sparing preferences.

27. The method of claim 22, wherein the trainee feedback comprises one of a beam energy and angle selection, a dose-volume selection, or a total dose and fraction selection based on knowledge data.

28. The method of claim 22, wherein receiving input indicating the action comprises receiving, via the user interface, one of a beam angle selection, a dose-volume selection, or a total dose and fraction selection for application to the target volume.

29. The method of claim 28, wherein determining trainee feedback comprises determining trainee feedback based on the one of a beam energy and angle selection, a dose-volume selection, or a total dose and fraction selection.

30. The method of claim 22, wherein the trainee feedback comprises visual feedback comprising a dose-volume histogram (DVH) curve, three-dimensional dose distributions of the subject and the derivate such as normal tissue complication probability, tumor control probability, radiobiological equivalent dose etc., computed from one or more training models within the system

31. The method of claim 22, wherein the response input comprises selection of a point on the DVH curve.

32. The method of claim 31, wherein the trainee feedback comprises indication of another point on the DVH curve.

33. The method of claim 31, wherein the trainee feedback comprises indication of normal tissue complication probability (NTCP).

34. The method of claim 22, wherein the target volume comprises liver, lung, kidney, brain, neck, stomach, pancreas, spine, bladder, and/or rectum.

35. The method of claim 22, wherein the one or more organs at risk comprises heart, lung, kidney, liver, brain, neck, stomach, pancreas, spine, bladder, and/or rectum.

36. The method of claim 22, further comprising:
presenting, via the user interface, the analysis data of the at least one parameter of the action;
receiving, via the user interface from the trainee, request regarding the analysis data; and
presenting, via the user interface, the most appropriate feedback for the trainee regarding the at least one parameter of the action.

37. A system comprising:
a computing device including a user interface and a radiation treatment planner training module configured to:
receive case data including anatomical and/or geometric characterization data of a target volume and one or more organs at risk;
present, via the user interface, the case data to a trainee;
receive, via the user interface from the trainee, input indicating an action to apply to the case, the input indicating one or more parameters for generating a treatment plan that applies radiation dose to the target volume and one or more parameters for constraining radiation to the one or more organs at risk;
apply training models to the action and the case data to generate analysis data of at least one parameter of the action; and
present, via the user interface, the analysis data of the at least one parameter of the action.

38. A system comprising:
a computing device including a user interface and a radiation treatment planner training module configured to:
receive case data comprising subject disease information, other medical condition information, and/or anatomical and/or geometric characterization data of a target volume and one or more organs at risk of a subject;
provide identification of one or more treatment plan analysis points for the subject;
provide knowledge data about treatment of the subject at the one or more treatment plan analysis points;
present, via the user interface, the case data to a trainee and the identified one or more treatment plan analysis units, and questions regarding the units;
receive, via the user interface from the trainee, response input to presentation of the one or more treatment plan analysis points; and
in response to receipt of the response input:
generate analysis data of trainee feedback based on knowledge data and the response input; and
present the analysis data via the user interface to the trainee.
